(19) United States
(12) Patent Application Publication
(10) Pub. No.: US 2009/0062697 A1
(43) Pub. Date: Mar. 5, 2009

(54) INSERTABLE ULTRASOUND PROBES, SYSTEMS, AND METHODS FOR THERMAL THERAPY

(75) Inventors: Jimin Zhang, Bellvue, WA (US); David M. Perozek, Mercer Island, WA (US); Lee Weng, Bellevue, WA (US)

Correspondence Address: KNOBBE MARTENS OLSON & BEAR LLP, 2040 MAIN STREET, FOURTEENTH FLOOR, IRVINE, CA, 92614, US

(73) Assignee: Therus Corporation, Seattle, WA (US)

(21) Appl. No.: 12/202,195

(22) Filed: Aug. 29, 2008

Related U.S. Application Data
(63) Continuation of application No. 10/671,417, filed on Sep. 24, 2003, now abandoned.
Application No. 10/671,417 is a continuation-in-part of application No. 09/696,076, filed on Oct. 25, 2000, now Pat. No. 6,656,136.
(60) Provisional application No. 60/413,118, filed on Sep. 24, 2002.
(60) Provisional application No. 60/163,466, filed on Oct. 25, 1999.

(51) Int. Cl. A61N 7/02

(52) U.S. Cl. 601/3

(57) ABSTRACT

Disclosed herein are methods and systems for producing hemostasis, tissue closure, or vessel closure by inserting a thermal delivery probe into a passageway and emitting thermal energy from the probe to produce the hemostasis or tissue closure. These methods and systems may be used following a percutaneous medical procedure that creates a passageway in tissue of patient, such as is caused by introduction of an access device into the patient. The thermal delivery probe may have one or more ultrasound transducers positioned in an elongated shaft.

### FIELD OF INVENTION

The present invention relates generally to the field of medicine and in particular to therapeutic devices and methods for delivering thermal energy to predetermined tissue volumes.

### BACKGROUND OF THE INVENTION

The use of thermal energy in the medical field for therapeutic purposes, specifically to induce tissue coagulation, necrosis, ablation, and various other tissues modifications, such as shrinkage or tightening, is well known. For example, YAG-lasers have been used to apply intense thermal energy to tissues to induce coagulation and to cauterize tissues. Various microwave, radiofrequency, light energy, and laser devices have also been developed to thermally treat tissues, in order to destroy malignant and benign cells and tissues, in a wide variety of body locations. A clear disadvantage of such treatments is that energy delivery is not well targeted and trauma is often sustained at unintended tissue locations during delivery; moreover, these techniques typically require the tissue in question to be in very close proximity to the delivery device. Other minimally invasive or non-invasive energy delivery methods and devices, which can be used to deliver targeted energy to specific tissue locations, are needed.

A growing number of medical procedures involve the percutaneous introduction of medical instrumentation directly into a vessel or into a patient's organ. To introduce such instrumentation, typically, an access device (such as an introducer sheath or a cannula) will enter the patient from a puncture site on the patient's skin, creating a passageway, or channel (referred herein as a tissue track), in the subcutaneous tissues. When the access device is removed, bleeding often occurs and the sealing (hemostasis) of, and subsequent healing of punctures or wounds caused during the procedure must be addressed. Examples of various medical procedures involving the introduction of instrumentation into a patient include percutaneous coronary, peripheral vascular, and neurovascular transcatheter procedures, tissue biopsy procedures, as well as needle biopsy procedures on organs. Although manual compression has proven successful in causing hemostasis and/or closure after such procedures, there are a number of problems associated with it. A non-invasive, or minimally invasive, method for thermally inducing protein denaturation, in order to seal the bleeding vessel and/or tissue track, is an ideal method of treatment.

Ultrasound technology is conventionally used for therapy and may be used as a means of satisfying the needs identified above. Applied in the appropriate operational conditions, sound waves can be used to selectively deposit thermal energy on tissues sites. In focused systems, beam intensities may increase along the emitted acoustic wave, with the highest intensities found at or near the “focus” of a therapeutic transducer. The intended bio-effects are caused in the tissues located within the “focal area” at a corresponding “focal depth” from the transducer. Focal characteristics and frequencies typically will determine where the maximal intensities are located. Moreover, depending on the operating parameters and design, the intervening tissues usually show little or no significant damage, since the energies reaching them can be controlled to be fairly low. The spatial placement of the lesion, as well as the concentration of the ultrasound beams, may be controlled using various techniques including electronic phasing and other steering techniques; use of acoustic lenses and cones; use of transducers having different or varying shapes and configurations; and choice of operating frequency of the transducer used for therapy. Ultrasonic energy deposition is an effective technique of volumetrically treating a pre-selected area of tissues.

The present invention describes the use of therapeutic ultrasound as a method for delivering thermal energy for a range of therapeutic applications, including for the following: hemostasis; vascular, and tissue, wound closure and sealing, including that required following a percutaneous medical procedure; focal ablation; venous valve tightening; and the treatment of female stress incontinence through tissue modification.

### SUMMARY OF THE INVENTION

Considered most broadly, the present invention is directed to the ultrasonic delivery of thermal energy to tissues in order to cause tissue necrosis, ablation, coagulation and/or shrinkage. In accordance with this aspect of the present invention, a method for delivering thermal energy to the tissues comprises the following steps of: inserting means for heating tissues percutaneously into the body of a patient; determining one or more sites to which thermal energy should be applied; emitting sufficient thermal energy to the site in order to raise native tissue temperatures; and inducing a pre-determined therapeutic affect.

In accordance with yet another aspect, the present invention describes methods and devices for acoustically sealing tissue tracks, vessel punctures and wounds, as well as for inducing hemostasis. A method for producing hemostasis, tissue closure, and/or vessel closure following a percutaneous medical procedure, wherein an access device has been introduced into a patient, creating a passageway, is described. This method is comprised of the following steps: (1) the insertion a thermal energy delivery probe into the passageway; (2) the determination of the site at which thermal energy should be applied; (3) raising native tissue temperatures by depositing sufficient thermal energy to the site; and (4) inducement of tissue and/or blood coagulation at the site.

The determination of the thermal delivery site is comprised of ultrasonically interrogating a section of the passageway using pulsed Doppler. This passageway may have been created in order to access a femoral vessel and, the vessel to be closed may be a femoral, brachial or peripheral vessel. Accordingly, the probe may be adaptively sized from typically about 2-7 French or larger.

In accordance with yet another aspect of the present invention, a method for producing hemostasis and tissue closure following a percutaneous medical procedure wherein an access device is introduced to a patient creating a passageway is described. Said method comprises the following steps of: (1) inserting an ultrasound probe into the passageway; (2) determining a site at which thermal energy should be applied; (3) emitting sufficient high intensity focused ultrasound energy to the site in order to raise native tissue temperatures; and (4) inducing tissue and/or blood coagulation at the site. The determination of the site at which thermal energy should be applied further comprises ultrasonically interrogating a section of the passageway using pulsed Doppler.

In accordance with yet another aspect of the present invention, various medical probes adapted to be inserted into a tissue passageway following a percutaneous medical procedure are described. These probes are generally comprised of: an elongated shaft having a proximal section, a distal section, a distal tip, and at least one lumen extending longitudinally from said distal tip to a proximal end located in the proximal section; a means for locating and determining a site at which thermal energy should be applied to promote hemostasis; and a means for emitting sufficient thermal energy to the site thereby raising native tissue temperatures in order to induce tissue and/or blood coagulation.

In accordance with yet another aspect of the present invention, various ultrasound insertable probes for delivering thermal energy are described. These probes are generally comprised of: an elongated shaft having a proximal end, a distal end, and at least one lumen extending longitudinally from said proximal end to said distal end; and one or more ultrasound transducers positioned in the elongated shaft. The one or more ultrasound transducers may be comprised of at least one therapeutic ultrasound transducer configured to emit high intensity ultrasound. The emitted thermal energy may be ultrasonically applied using a high frequency, high power output, ultrasound transducer. This high frequency, high power output ultrasound transducer may be located at a distal end and/or proximal end or section of the thermal delivery probe. As is described in further detail below, the high frequency, high power output ultrasound transducer may be operated at about 6 MHz and output about 2 W. These insertable ultrasound probes may also be further configured to emit low-intensity, diagnostic ultrasound, and adapted to ultrasonically interrogate a position in front of the elongated shaft distal end.

For a better understanding of the present invention, together with other and further objects, reference is made to the following descriptions, taken in conjunction with the accompanying drawings.

### BRIEF DESCRIPTION OF THE DRAWINGS

FIG. 1A is a schematic depiction of a therapeutic ultrasound system in accordance with the present invention.

FIG. 1B is a system block diagram depicting a therapeutic ultrasound system in accordance with the present invention.

FIGS. 2A-2D are schematic depictions of an insertable probe in accordance with the present invention wherein:

FIG. 2A is a perspective view of an insertable probe of the present invention;

FIG. 2B is a partial perspective view of the insertable probe taken along lines 3-3; and

FIG. 2C is a partial longitudinal cross-sectional view of the distal section and distal tip of the insertable probe taken along lines 9-9.

FIG. 2D is a schematic illustrating one method of positioning insertable probe inside a patient.

FIGS. 3A-3B are a perspective view of yet another embodiment of an insertable probe of the present invention wherein:

FIG. 3A is a schematic illustrating a vascular wound being percutaneously and thermally sealed; and

FIG. 3B is a partial longitudinal cross-sectional view of insertable probe illustrated in FIG. 3A.

FIG. 4 is a perspective view of a guidewire adapted insertable probe in accordance with this aspect of the present invention being advanced to a treatment location.

FIG. 5 is a partial longitudinal cross-sectional view of the insertable probe shown in FIG. 4.

FIG. 6 is a partial longitudinal cross-sectional view of yet another embodiment of an insertable probe incorporating an additional heating system in accordance with this aspect of the present invention.

FIG. 7A is a schematic depiction of a preferred embodiment of the present invention.

FIG. 7B is a partial longitudinal cross-sectional view of the distal tip of the insertable probe illustrated in FIG. 7A.

FIG. 7C illustrates broadly a method of appropriately delivering the insertable probe of the present invention inside the patient.

FIG. 8 is a flow chart diagram illustrating a preferred method of using the probe embodiment depicted in FIGS. 7A-7C.

### DETAILED DESCRIPTION OF THE INVENTION

Referring to the drawing figures, like reference numerals designate identical or corresponding elements throughout the figures.

Broadly, the present invention is directed at delivering targeted thermal energy to tissues. The basic concept of the present invention involves the insertion of probe 8, adapted to emit thermal energy, and its advancement into an operative location inside the patient's body. Using various methods, the targeting and application of an appropriate thermal dose to the appropriate tissue treatment site is accomplished. Absorption of energy by the tissues will raise the native tissue temperatures and when sufficiently high temperatures and exposure times are reached, will establish a coagulum and “biological glue,” which will either act to seal the wound or to close a tissue tract and/or vessel opening, and/or tissue tightening. After confirming the completion of the therapeutic treatment (i.e., cessation of bleeding or closure of the vessel or tissue track) the inserted probe may be withdrawn. During, or after, the treatment, the operator may apply manual pressure to the treatment site to impede bleeding and promote the efficiency of the therapeutic treatment. Various diagnostic, ultrasound techniques may be used to direct the insertion of probe 8 and deliver, target, and appropriately apply, the correct dosage, or exposure, of therapeutic ultrasound.

### System

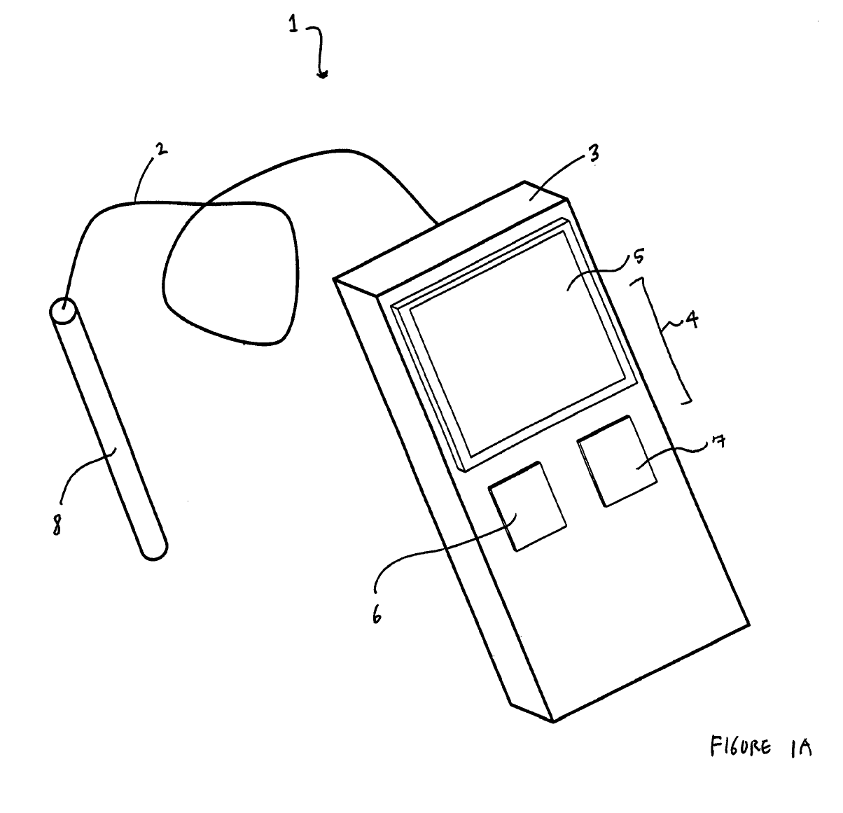

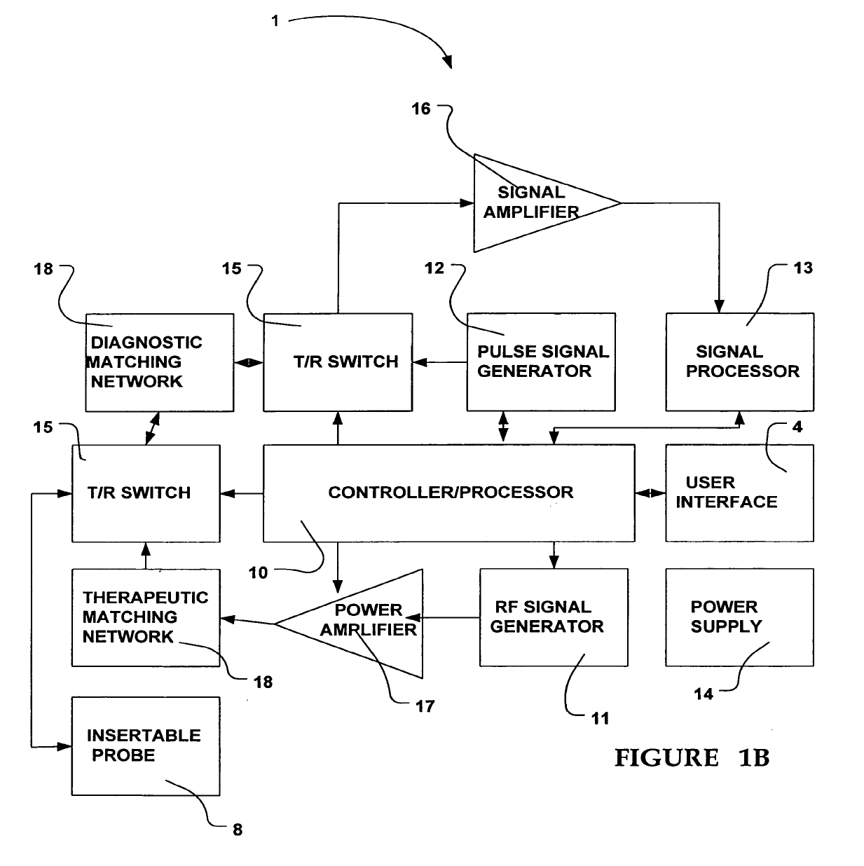

FIGS. 1A and 1B illustrate a typical therapeutic ultrasound system 1 in accordance with the present invention. Therapeutic ultrasound system 1 is comprised of probe 8, having one or more ultrasound transducers 25, which are operationally interconnected, by cable assembly 2, to the various system components, which are housed in device 3. Preferably, device 3 is configured to be portable and is comprised of one or more visual displays 5, controls 6, indictors 7, keyboards and/or various buttons to facilitate ease of use and operation. System 1, buttons, and controls 6 of device 3 should be configured to allow for various user inputs, including, for example, user commands to “power on” system 1 and to initiate a therapeutic treatment protocol.

FIG. 1B illustrates the potential connections between the various system components and the one or more ultrasound transducers 25 located in probe 8 in a block diagram. In this diagram, system 1 is configured with both diagnostic and therapeutic ultrasound functionality. Using various Doppler and echo amplitudes, the diagnostic capability of the present invention allows the system user to correctly advance and deliver probe 8 to an operative location inside the patient. It also determines the appropriate treatment site for targeting the therapeutic ultrasound beams, while also confirming sufficient exposure of the tissue to the ultrasonically delivered thermal energy, and the completion of the treatment.

As depicted in FIG. 1B, system 1 may be comprised of one or more of the following components: a controller/processor 10; a RF signal generator 11; a pulse signal generator 12; a signal processor 13; a power supply 14; user interface 4; a transmit and receive (T/R) switch 15; a signal and power amplifier 16, 17; drive electronics; matching or impedance networks 18; and other conventional ultrasound system components. The controller/processor 10 may be a microprocessor that communicates with the user interface 4. To appropriately drive one or more of the ultrasound transducers 25 through the various impedance or matching/tuning networks 18, the controller/processor 10 generates and transmits the necessary timing and control signals to the signal processor 13, the RF signal generator 11, and the pulse signal generator 12. In addition, one or more of the T/R switches 15 may be employed to gate on and off the electronic signals from either the controller/processor 10 or the pulse signal generator 12, or RF signal generator 11, or signal processor 13 to the one or more ultrasound transducers 25. Power amplifiers 17 may be provided in order to boost the signals generated by the RF signal generator 11 and pulse signal generator 12, as well as drive the one or more of the transducers 25 so that each transducer 25 emits the appropriate amount of acoustic energy. Signal amplifiers 16 may be incorporated in system 1 to amplify and improve the received signals from the ultrasound reflected back from the tissues.

Turning now to the other drawing figures, various embodiments of probe 8 devices, in accordance with this aspect of the present invention, will be described. As will be appreciated by those skilled in the art, the various probes 8 may be adapted and modified for specific therapeutic applications.

### EMBODIMENT 1

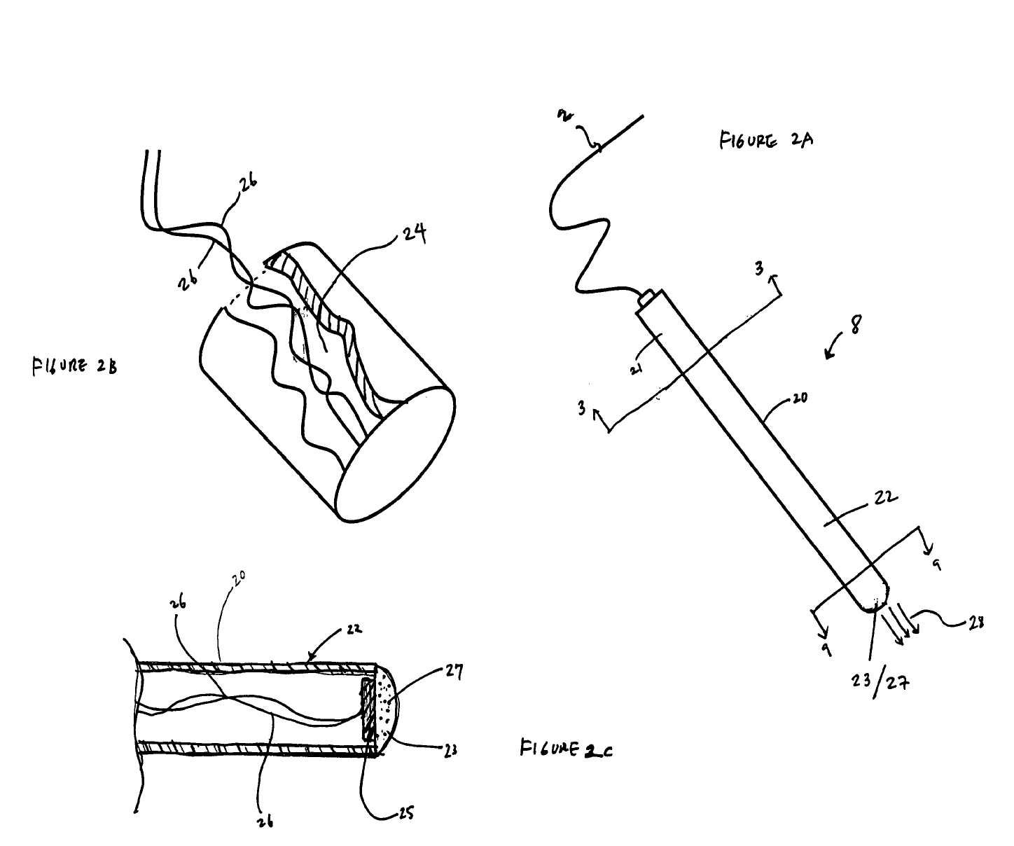

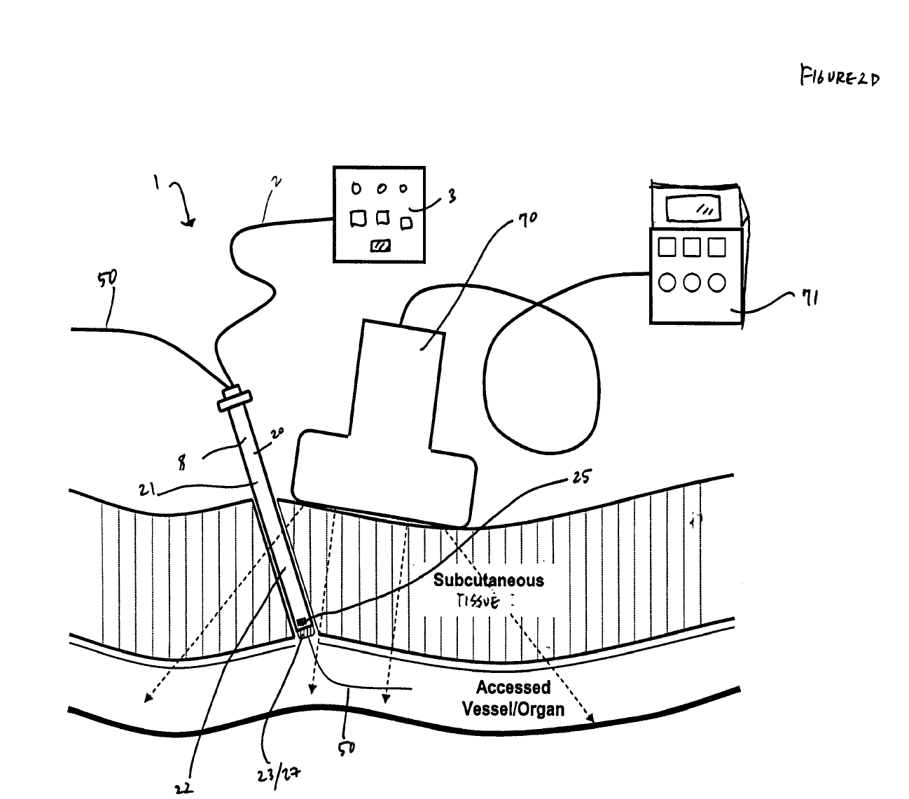

FIGS. 2A, 2B and 2C illustrate one embodiment of insertable probe 8. As best illustrated in FIG. 2A, insertable probe 8 is comprised of an elongated shaft 20. Elongated shaft 20 is comprised of proximal section 21, distal section 22, and distal tip 23, located at the distal extremity of distal section 22. Elongated shaft 20 is generally a hollow tube comprising a lumen 24 and is generally adapted to house ultrasound transducer assembly 25, as well as any and all other various electronic connectors, cables, and/or wires (collectively illustrated by reference no. 26) needed to operationally interconnect transducer assembly 25 to the rest of therapeutic system 1 and device 3. Alternatively, shaft 20 may be comprised of a solid materials and the various other component being embedded in said material.

Elongated shaft 20 may be fabricated from any thermally conductive material having high specific heat characteristics (such as copper, brass, nitinol, or other like materials), and may be disposed with one or more lubricious materials to facilitate advancement of probe 8. Preferably, elongated shaft 20 should be configured to be sufficiently flexible for navigation inside the patient's body, yet sufficiently stiff to allow its advancement to the operative location.

In this embodiment, transducer assembly 25 is disposed in distal section 22 of elongated shaft 20. Transducer assembly 25 of the present invention may include any number of ultrasound transducers including, but not limited to, separate diagnostic and therapeutic transducers, or a single transducer capable of being operated in both diagnostic and therapeutic ultrasound modes. As will be appreciated by those skilled in the art, the selection of the types, size, and shape of the ultrasound transducers to be used will typically be based on the intended therapeutic application, design considerations, among other conditions. Any ultrasound waves generated by transducer assembly 25 are emitted from distal tip 23 of probe 8. Distal tip 23 may be further adapted to include one or more acoustic lens 27. Acoustic lens 27 promotes the efficient transmission of acoustic beams 28 from transducer assembly 25 to the tissues. It also provides mechanical protection for probe 8; electrical isolation; assists in geometrically shaping the emitted acoustic beam; and can be adapted to promote the advancement of probe 8 into the patient's body without trauma.

In the present invention, the delivery of distal tip 23 to an operative location is important in ensuring that the emitted acoustic energies will reach the intended treatment site, and ensuring that unintended tissues are not treated. FIG. 2D illustrates one method of directing probe 8 into the correct treatment location and position. This method uses a conventional ultrasound imaging system 71, having a handheld applicator 70, in conjunction with probe 8. As illustrated in FIG. 2D, applicator 70 is used transcutaneously and provides a method for ultrasonically guiding the user and aiding in the correct placement of probe 8 inside the patient.

In addition to this method, various ultrasonic signal analysis techniques may also be used to ensure proper positioning and maintenance of probe 8 in the patient. At their core, these techniques involve subjecting the tissues to an ultrasonic interrogation beam; analysis of the reflected beam, or returning ultrasonic signal; and the comparison of any ultrasonic variation between the emitted and reflected beams, or signals. The time variation of the reflected beams, or signals, may be used to map the internal anatomy, or structure, of the tissues; and to “observe” any changes in tissue state, or some other predetermined variable that may indicate critical treatment parameters and dictate treatment.

The information provided by the signal analysis may also be used to create a graphical depiction of the anatomy and/or structure of the tissue. This graphical depiction may alert the system operator when the probe 8 is in position, and when therapy may be initiated and/or ended.

These ultrasonic signal analysis techniques may be automated and integrated into system 1 and different methods can be used to alert the user to the outcomes of these analyses. These alerts may include auditory alerts; lighted signals; a user interface 4; and/or other messaging or communication means. Further detail of this aspect of the present invention is provided below, with specific reference to the sealing of a vessel puncture or wound.

### EMBODIMENT 2

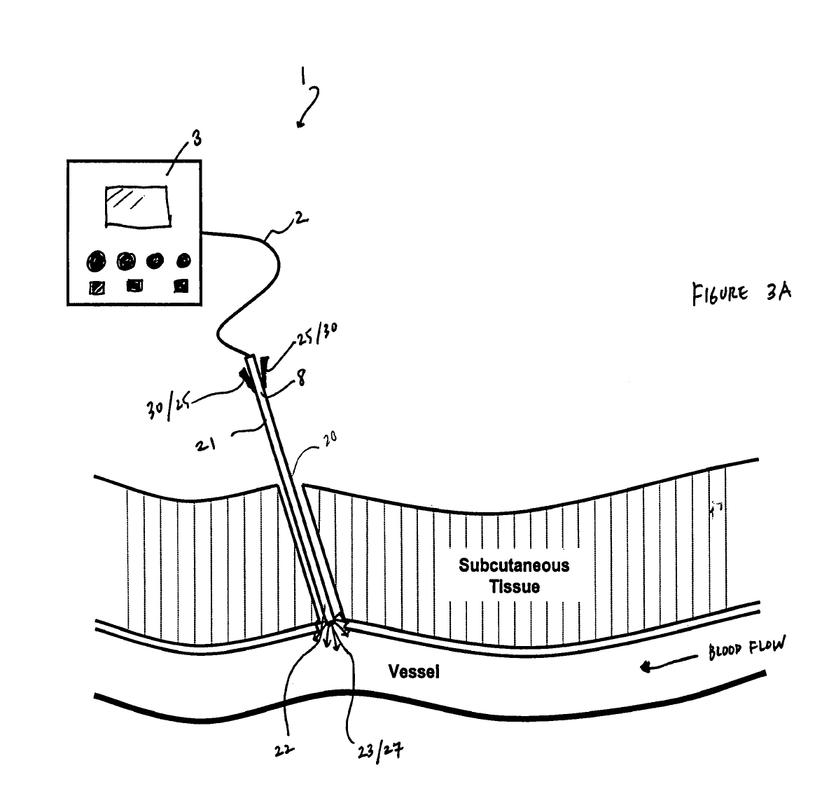

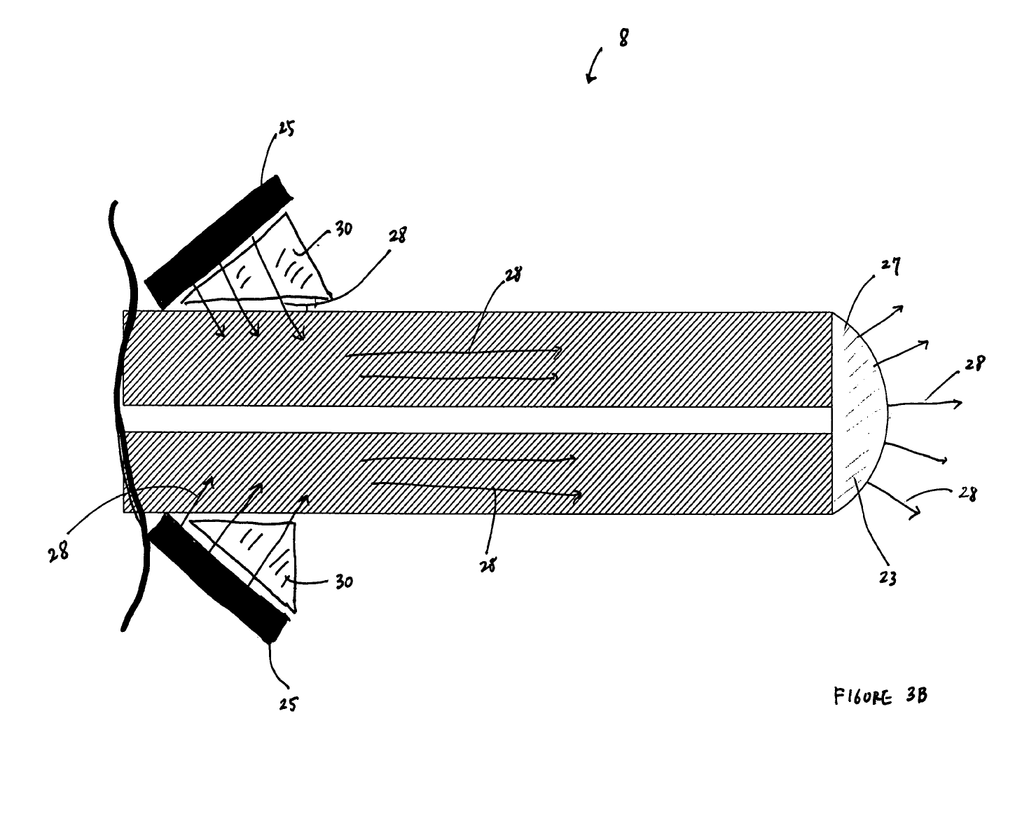

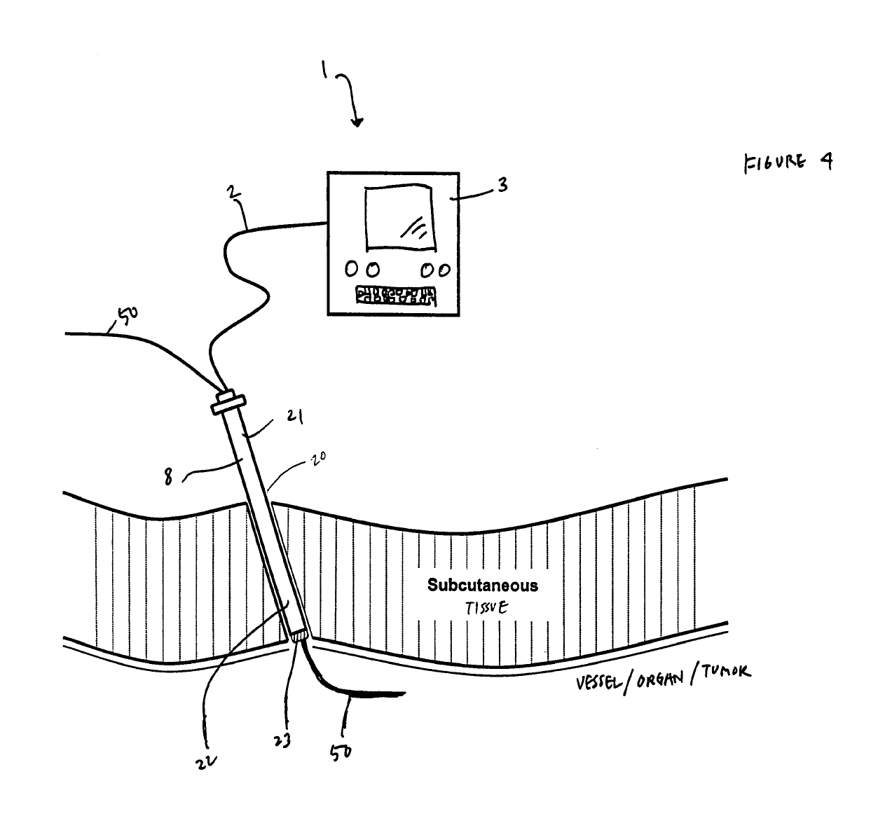

FIGS. 3A-3B illustrate another embodiment of probe 8 wherein a “wave guide” design is used. In this embodiment, the emitted acoustic beams from transducer assembly 25, located at proximal section 21, are transmitted, or guided, down elongated shaft 20. The acoustic waves (depicted by arrows), generated by transducer assembly 25, are propagated down elongated shaft 12 and emitted from the distal tip 23 to the tissues.

As shown, transducer assembly 25 is coupled to elongated shaft 20 via a stand-off means 30 outside of, or external to, elongated shaft 20. As will be appreciated by those skilled in the art, stand-off means 30 and elongated shaft 20 should be fabricated from materials with low attenuation characteristics in order to allow for the efficient transmission of acoustic waves into, and down, elongated shaft 20. However, the acoustic velocity of these materials should differ, thus allowing the emitted acoustic waves (depicted by the arrows) to be bent, or refracted, so that they are propagated down elongated shaft 20 and emitted from distal tip 23 through acoustic lens 27. (See FIG. 3B.) Standoff means 30 may be fabricated from polystyrene, plexiglass, aluminum, titanium, or other similar materials. Similarly, elongated shaft 20 may be fabricated from quartz, aluminum, titanium or other like materials.

In the present embodiment, illustrated in FIGS. 3A-3B, any high power transducer(s) may be used to generate the necessary therapeutic acoustic beams. Any high power, single element or multi-element, linear or phased, transducer array may be used, including a single high power array or various stacked array configurations. These arrays may be fabricated from piezoelectric ceramic (PZT), composite materials, or other like materials. A good review of acoustic wave generation using “wave-guide” designs is provided in Rose, Joseph L. 1999. Ultrasonic Waves in Solid Media. New York: Cambridge University Press. Chap. 14 & E4, the entire contents of which are hereby incorporated by reference.

### EMBODIMENT 3

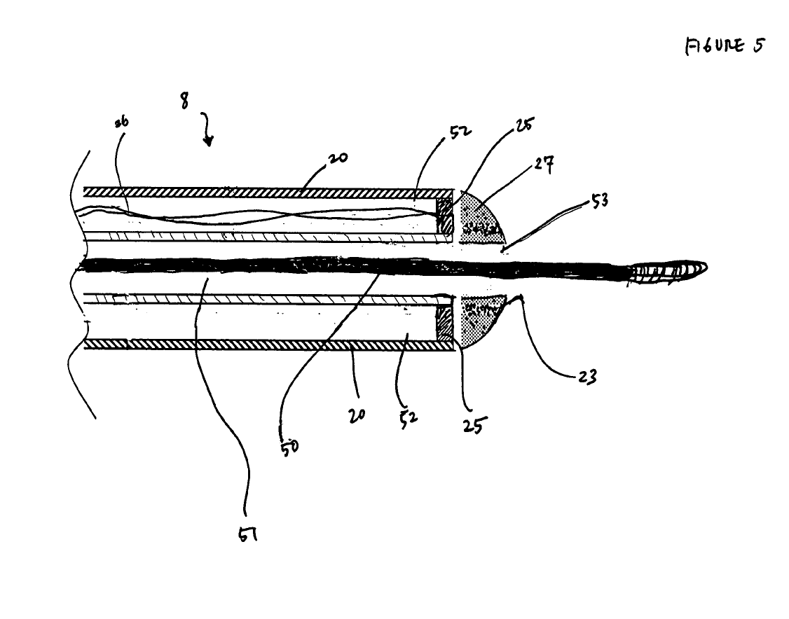

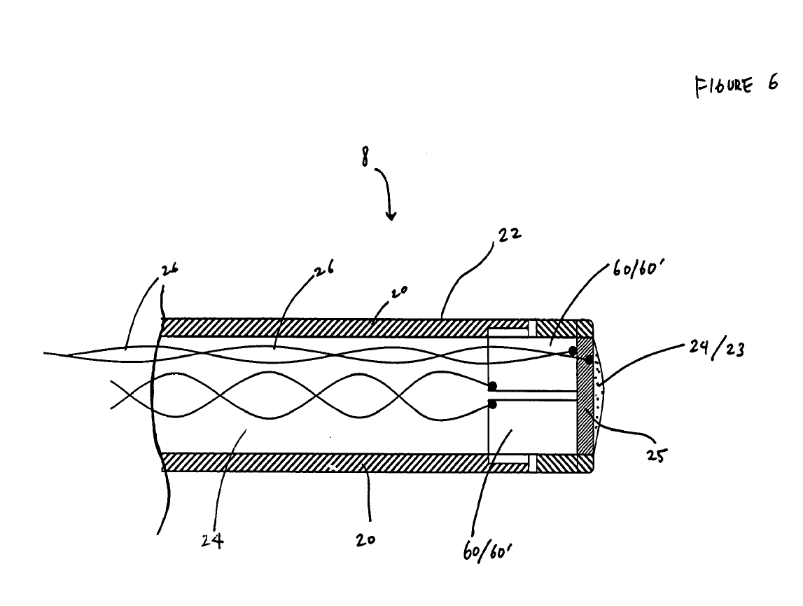

Referring to FIGS. 4 and 5, an additional embodiment of insertable probe 8 is provided. In this embodiment, probe 8 is adapted for use in conjunction with guidewire 50 (a device typically used in various percutaneous coronary, peripheral, and neurovascular transcatheter procedures). In this embodiment, probe 8 is comprised of one or more lumens. In FIG. 5, a dual lumen configuration is shown wherein inner lumen 51 and outer lumen 52 extend longitudinally from distal tip 23 of elongated shaft 20 to the proximal end or section 21 of elongated shaft 20. Inner lumen 51 is provided so that guidewire 50 (or other like device) may be threaded through it and used to advance probe 8 to an operative location inside the patient. Inner lumen 51 is comprised of a first exit port (not shown) positioned at proximal section 21 of elongated shaft 20 and a second exit port 53, preferably positioned in distal shaft section 22. These ports allow for the guidewire to be threaded through and out probe 8.

### EMBODIMENT 4

Referring now to FIG. 6, another embodiment of probe 8 is illustrated. In this example, heating means 60 (in this case a split conductive ring 60′) may be incorporated at distal tip 23 of probe 8. This conductive ring 60′ may be any electrically driven heat resistor or other like heating means. Electrically supplied heat, or thermal energy, may be applied in conjunction with acoustically delivered thermal energy to enhance absorption of acoustic energies in the tissues. As will be appreciated by those skilled in the art, the application of energy from another heating source, or system, can be advantageous to the present treatment, as less energy output is required of transducer assembly 25 to raise native tissue temperatures acoustically to the appropriate therapeutic levels.

As is known by those skilled in the art, excessive heat generation can pose issues related to the sticking of heated tissues and coagulated blood components to probe 8. In order to minimize and/or eliminate this issue, several strategies may be employed. For example, the various probes 8 of the present invention may be fabricated from various lubricious, non-adhesive, biocompatible materials. Similarly, the exterior surface of the various probes 8 may be covered (using dipping, extrusion, vapor deposition, or sputtering methods) by one or more compounds that are lubricious, non-adhesive, and biocompatible. Examples of this material include various hydrogels, polytetrafluoroethylene (PTFE), expanded polytetrafluoroethylene (ePTFE), perfluoro(propylvinylether) (PFA), polyethylene, various co-polymers and blends of polyethylene, polyethylene block amide, polyesters, polyurethanes, polyamides, nylon, and mixtures thereof.

Moreover, and as will be appreciated by those skilled in the art, variations, enhancements, and modifications of the above described embodiments of system 1 and probe 8 are possible. For example, probe 8 may be adapted to provide the delivery of therapeutic compounds, such as pain control agents or sealing accelerants (e.g. prothrombin), to the tissues prior to or after energy delivery. Additionally various tissue sealant compounds, such as various fibrin glues, albumin soldering materials, and other protein glues or sealing materials, may be introduced to the tissues to further enhance tissue and vessel closure and hemostasis. These therapeutic compounds may be introduced through a lumen in probe 8 configured for such purposes. Further details about the various types of sealing materials that may be used in the present invention are provided in Wolf, et al. “Comparison of Fibrin Glue, Laser Weld, and Mechanical Suturing Device for Laparoscopic Closure of Urterotomy in a Porcine Model,” J. Urol. 157 (1997); Trickett et al., In Vitro Laser Nerve Repair: Protein Solder Strip Irridation or Irradation Alone?, Int. Surg. 82 (1997); and Kirsch, et al, Laser Soldering Techniques for Sutureless Urethral Surgury, Tech. Urol. 3 (1997), the entire contents of which are hereby incorporated by reference.

Additional variations, enhancements and modifications to the above described embodiments of the present invention include the disposal of location sensors, temperature sensing means, or other like devices on distal section 22 of any of the insertable probes 8, to provide positioning, as well as monitoring, functionality to the present invention.

Cooling systems may also be incorporated into probe 8 of the present invention, to enhance its performance. For example, probe 8 may be adapted to include a water channel or cooling lumen to allow cooling fluids to be circulated through it or to allow the delivery of cooling fluids directly to the tissues.

Probe 8 may also be adapted for disposability or may be provided with a removable and disposable sheath. Various temperature sensing elements may be used to monitor temperature changes in the tissues, as well as the dosage exposure of the tissue to thermal energy. Temperature sensors, such as various thermocouples, thermistors, and/or infra-red sensing diode may be used and located at distal tip 23.

As will be described in further detail below, various automations and other features for locating and correctly positioning probe 8, may also be provided and operationally integrated into system 1. Any of these features, in addition to features discussed with respect to a particular embodiment, may be combined with any or all of the features of another embodiment, and all such combinations are within the scope of the present invention.

### EMBODIMENT 5

Yet another embodiment of the present invention (system 1 and probe 8), specifically adapted for vascular, or vessel, sealing applications (e.g. the treatment of punctured or wounded arteriotomies, including femoral; brachial; or other peripheral vessels) following a percutaneous transcatheter procedure, shall be described. The present approach to vascular, or vessel, sealing affects a seal in the punctured or accessed vessel by denaturing the protein in the vicinity of the wound or puncture. Denaturization is thermally accomplished through the absorption of high intensity ultrasound delivered to the treatment site by probe 8. The present description, and the accompanying drawings related to this embodiment, are provided as way of illustration and to describe various exemplary methods of the present invention. They do not, and are not intended to, in any way limit any aspect of the present invention.

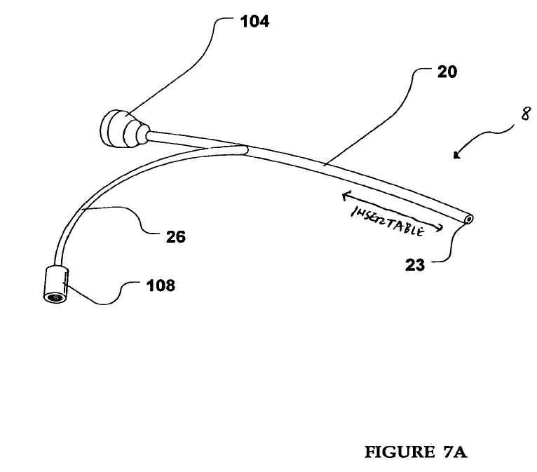

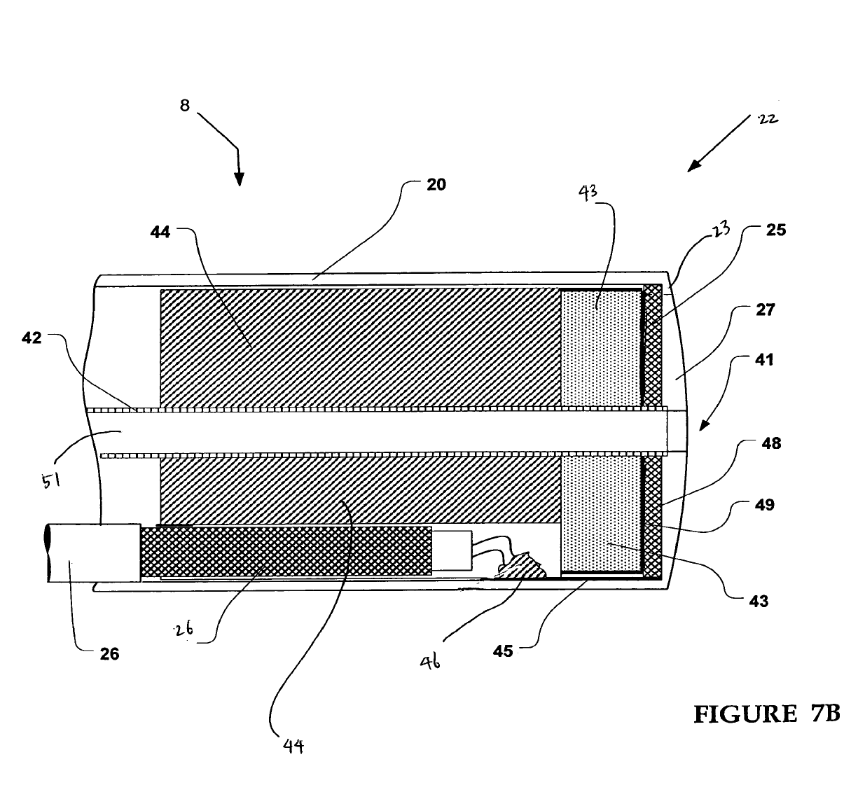

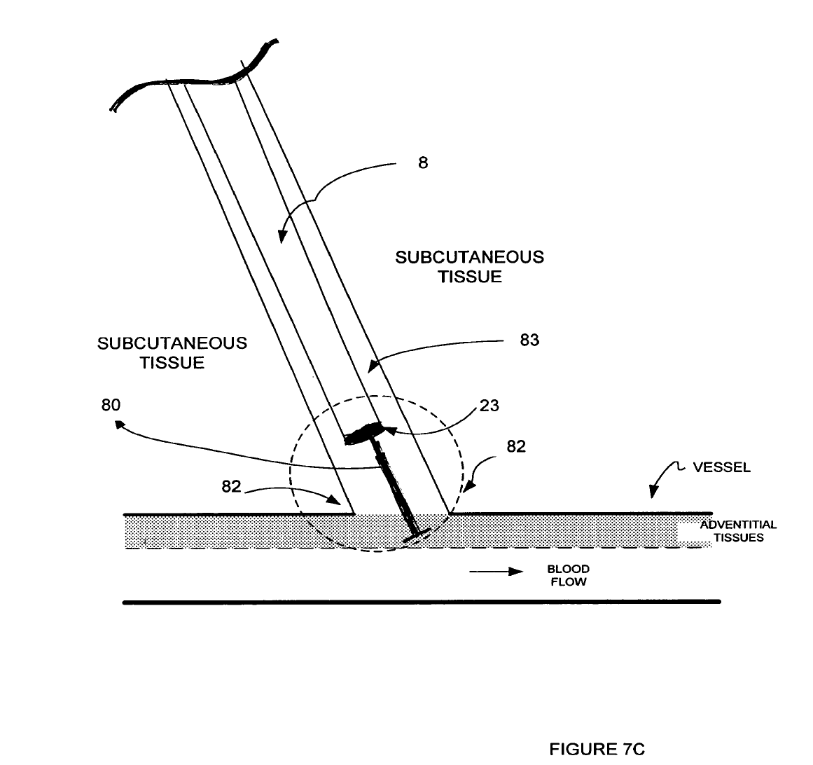

FIG. 7A illustrates one embodiment of a vascular sealing probe 8. As shown, probe 8 is comprised of elongated shaft 20, cable assembly 26 having cable connector 108, and gripping knob 104. Cable assembly 26 may be a flexible shield coaxial cable about 2 mm in diameter having a diameter of about 0.40 mm inside the probe, and about 30 cm long. Gripping knob 104 may be optionally provided to allow a user to hold onto probe 8 during operation and also to aid in the advancement of probe 8 into the body.

In this embodiment, the length of elongated shaft 20 is preferentially about 15 cm, with an insertable length of about 6.4 cm, as indicated in FIG. 7A. This size allows the probe to be used to access and treat vessels located as deep as approximately 4 cm from the surface of the skin. However, as will be understood by those skilled in the art, the specific dimensions provided herein may be altered and modified. Preferably, the diameter of elongated shaft 20 should be about 7 F (2.3 mm) or smaller. It should be semi-flexible and sufficiently pliable so that it conforms to any curvatures of the entry channel, or tissue track, but also sufficiently rigid to allow advancement of the probe into the patient without kinking. Elongated shaft 20 may be molded from PTFE or other like materials.

FIG. 7B illustrates in greater detail distal tip 23 of probe 8 illustrated in FIG. 7A. As illustrated, distal tip 23 is comprised of a disc shaped acoustic transducer 25, positioned and oriented such that the emitted acoustic beams are substantially axial and are emitted forward from distal tip 23.

Transducer 25 may be fabricated from PZT-4 or other like piezoelectric materials, preferably having a thickness of about 0.34 mm. It may also be a 3 mm OD disc with a 0.5 mm center opening (exit port) 41 disposed therein. One or more acoustic lenses 27 may be provided at distal tip 23 to focus the ultrasound beam from transducer 25. Lens 27 may be fabricated from PFTE, or other like material. However, the use of lens 27 is not required. Natural focusing of disk transducer 25 may be used to optimize focusing of the ultrasound beam. Thus, probe 8 may be configured with no lens 27 or a flat lens 27 with an essentially infinite radius of curvature.

The center opening (exit port) 41 of transducer 25 should be configured to be in communication with guidewire lumen 51 disposed within elongated shaft 20. Lumen 51 may be an internally insulated plastic tube 42 and insulated so that a guidewire 50 may be inserted longitudinally along the length of probe 8 and advanced out of the center opening (exit port) 41 of transducer 25 without guidewire 50 being in electrical conductive contact with the other components of probe 8.

Transducer 25 may be operated at a resonant frequency in the range of about 4-12 MHz (preferably 6 MHz), and may be electroded with one or more metal layers 48, 49. Transducer 25 may be air backed, but preferably will be backed with any high thermal conductively, low density material 43 (about 1 mm thick), such as POCOFOAM® (commercially available from POCO Graphite Inc. of Decatur, Tex.). Several advantages are provided by backing transducer 25 with a high thermal conductivity, low density backing material 43, including: the provision of thermal conduction away from the transducer to heat sink 44; the provision of a large acoustic mismatch and reflection of sound back toward the front surface of the transducer. With the addition of the backing material 43 the transducer 25 efficiency is about 50% and heat is generated. However, in the present embodiment, heat generated by transducer 25 during its operation will be partially conducted to the backing material 43, which will then be conducted through the elongated shaft 20 to the tissues in the entry track. The backing material 43 may be bonded with a thin, thermally conductive epoxy to both transducer 25 and heat sink 44.

As will be appreciated by those skilled in the art, heat sink 44 is provided and should be formed from a material having high specific heat, density and conductivity, such as copper or other like material. The dimension of an exemplary heat sink 44, for use with this embodiment, is about 3 mm in diameter and 1.5 cm in length. Heat sink 44 may be disposed longitudinally with elongated shaft 20 or in any other configuration to accommodate other devices and components. The shield of coaxial cable 51, about 0.5 mm in diameter, may be electrically connected to heat sink 44 and its center conductor, terminated at connection 46 to conductive foil tab 45, may be connected to electrode 48 and transducer 25.

In the present embodiment, illustrated in FIG. 7A-7B, when probe 8 and transducer 25 are operated at or about an operational frequency of 4-12 MHz, preferably 6 MHz, transducer 25's focus is from about 25-7.5 mm; and weakly focused so that the focal spot size is on the order of about 1 mm. The power range of the present embodiment is 0.5-4 acoustic Watts. Under these conditions sealing can typically be affected in less than 20 secs. In the present embodiment, transducer 25 may be operated in a continuous wave (CW) mode during the therapeutic treatment with interruptions allowed for targeting and/or exposure control interrogations, at intervals of about 1 sec.

As briefly described above, various diagnostic ultrasound modalities may be used to ensure correct position of probe 8 inside the tissue track. Once transducer 25 is energized, the emitted therapeutic acoustic energy and beams are supplied to the treatment site. FIG. 7C is provided to better illustrate these concepts. As illustrated, probe 8 should be advanced to an optimal position (or striking or target distance 80), and in geometric relationship to the vessel opening or wound. Preferably, distal tip 23 should be advanced until it is within appropriate treatment distance (for example, about 2 mm) from the outer, or adventitial, layers of the vessel to be treated. This ensures that thermal energy will be ultrasonically deposited in the appropriate vessel tissues in order to affect closure. It will also ensure that the ultrasonic beam will deposited between the tissue boundaries 82 depicted in FIG. 7C.

Several methods may be employed to position distal tip 23 the optimal distance from the vessel to be sealed or thermally treated. In one embodiment, an over the wire technique may be used wherein probe 8 is advanced down into the tissue track 83 to a position substantially near the vessel and tissue opening to be sealed. Then, various Doppler signals are used to interrogate the tissues and provide a positive indication to the operator of correct placement of probe 8 within the appropriate strike/target distance 80. For example, a high frequency (approximately 6 MHz), diagnostic amplitude (e.g. <500 mW average power) short pulse (less than 100 cycles) can be repetitively transmitted as the operator advances probe 8 into the patient. A Doppler shift signal may be processed from the return range gated echoes from the tissues such that a logical flag is set in system 1 when blood flow is detected at a pre-determined distance, on the order of 2 mm in front of distal tip 23. Thus, when system 1 is used to seal a wound in a blood vessel and probe 8 must be positioned adjacent, but external to, the vessel to be sealed, this pulsed Doppler interrogation system is effective in aiding in the correct advancement of probe 8 into the patient. The state of this logical flag may be displayed appropriately to the operator via the user interface 4, or alternatively, as an audio tone or other like signal. Therefore, correct positioning is accomplished by advancing probe 8 to a point where a Doppler signal (set flag) just begins. Using single line Doppler, a pulse of 8 cycles may be transmitted, 20 such pluses may be averaged requiring about 500 microseconds per Doppler line, providing adequate spatial resolution of about 1 mm.

In an yet another example, during advancement and positioning, probe 8 and the present system 1, may be configured to continuously send Doppler lines and measure the distances from the probe to the vessel (with blood flow) to be treated. When flow is detected within a predetermined, pre-set range of distances, system 1 may be configured to initiate an emission of therapeutic acoustic waves for thermal heat treatment and subsequent closure of the vessels.

Typically, a preset dose of about 2 acoustic Watts for about 20 seconds should be sufficient to affect hemostasis and vessel closure under the operational parameters described above. However, various techniques may be used to more precisely control the thermal affect of the present invention and apply the appropriate acoustic doses. Specifically, various closed-loop control methods may be used to control and modulate the thermal delivery process.

In closed-loop exposure control, therapeutic treatment is initiated as a series of CW epochs, or tone bursts, of high intensity ultrasound, sequenced with a series of A-mode tissue interrogations. The A-mode tissue interrogations may be applied using the same pulse parameters and ensemble as described above. Generally, the interrogation parameters at each interval should be compared to values logged prior to the initiation of treatment. Precise dose control is achieved by ceasing the application of therapeutic power, or energy, when a monitored parameter reaches a value indicative of tissue state change and/or, in this case, effective sealing. Such closed-loop control may also be achieved by controlling the time that the therapeutic energy is applied or controlling the level of therapeutic power.

For example, the amplitude of the return interrogation signals should be measured and averaged over 64 ensembles to obtain sufficient signal-to-noise measurements. Amplitude may be measured at a spatial region about 1 mm long, located about 1 mm prior to the point at which blood flow is first detected. The amplitude should be recorded prior to initiation of the thermal treatment and at intervals throughout the treatment, for example at every 1 second interval. (Treatment will be a series of thermal treatments and interrogation epochs.) In accordance with this method, as time progresses, the differences in amplitude should be calculated and, if these differences exceed a pre-determined value for a specified time, the treatment may be terminated.

As will be understood by those skilled in the art, the amplitude effectively represents the change in the acoustic absorption coefficient, which is a function of temperature and of the treated tissues. The changes in amplitude will indicate the extent of the heating of the tissues being thermally treated, allowing the therapeutic treatment to be measured as a function of time and/or temperature. These interrogation techniques, and the principles embodied therein, may be used to ultrasonically monitor any type of therapeutic treatment, and they are not limited to practice solely in the context of vascular sealing.

And finally, Doppler interrogation may be used to assess that vessel sealing has been achieved by attempting to detect bleeding. If bleeding were detected, an additional treatment dose could, at the operator's election be administered or, the system could do so automatically.

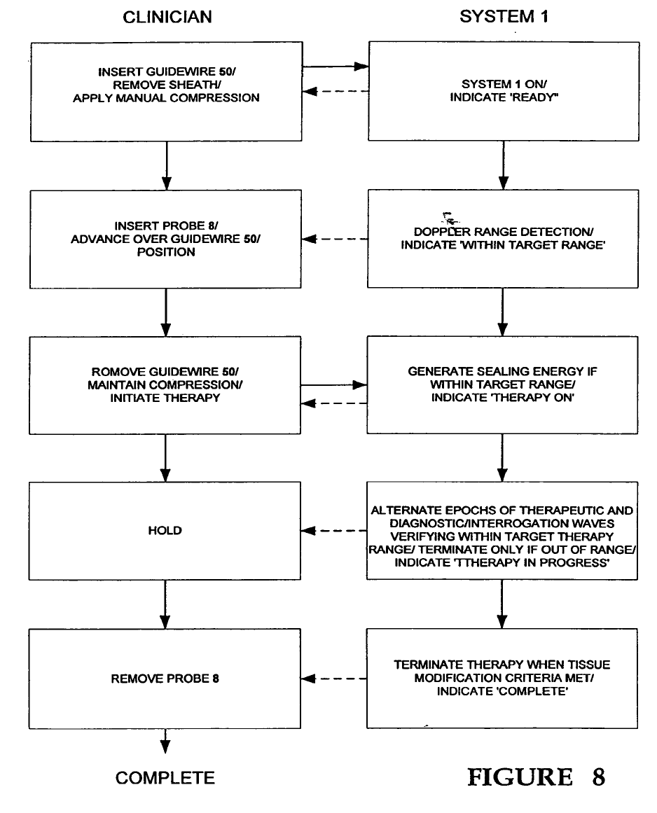

FIG. 8 is provided to schematically illustrate a method in accordance with the present invention of using insertable probe 8 and system 1 to ultrasonically to affect vessel sealing and induce hemostasis. In the method example provided in FIG. 8, it is assumed that the treatment method is being performed following a percutaneous trancatheter procedure wherein an introducer sheath device is still disposed within a patient and is used to access a vessel and the present invention will be used to ultrasonically seal this vessel thereby inducing hemostasis, as well as tissue track 83 and vessel closure.

### Transducers

As will be appreciated by those skilled in the art, various ultrasound transducers and transducer assemblies may be incorporated into probe 8 to affect therapeutic heating, as well as diagnostic interrogation. For example, various microelectromechanical (“MEMS”) ultrasound transducers may be used and incorporated into any of the probes 8 and systems 1 described above. MEMS transducers provide for high power densities and can be fabricated at low costs and in large volumes. These MEMS transducers may be operationally located at distal tip 23 of probe 8 to affect the emission of the appropriate therapeutic dosage of acoustically delivered thermal energy. These transducers may be operated for therapy (high output) and imaging, or Doppler, modes. Because these transducers can be fabricated at relatively low costs, probes 8 incorporating these transducers may be made as single-use, disposable probes 8. Single-use, disposable probes 8 ensure sterility of the therapeutic application, improve ease of use, and alleviate demanding service-life requirements of probes 8. Further details, as well as methods for making MEMS transducers, are provided in the following references, which are hereby incorporated by reference in their entirety: Percin et al., Micromachined Two-Dimensional Array Piezoelectrically Actuated Transducers, 7 Applied Physics Letters 11; (1998) and Ciftadine, A., MEMS Reshapes Ultrasonic Sensing, Sensors, (February 2000).

Various single element or multi-element transducer arrays may be used in transducer 25. The ultrasound transducer arrays may be a phased linear or phased annular array and should be driven, for example, by the appropriate drive electronics in order to generate the necessary acoustic intensities to affect the desired tissue change. Frequency modulation of power applied to the array may be used to shape the thermal lesion (e.g., a lower frequency may be used to establish a lesion at a first depth and an increasingly higher frequency may be established to create a lesion at a second, different depth, and conductively heating the tissues between the first and second depths). As will be appreciated by those skilled in the art, the selection of a specific and appropriate transducer (e.g., single or dual functionality transducer, a separate diagnostic and therapeutic transducer, phased or linear) and transducer assembly, along with the associated components, should be determined by the specific therapeutic application, as well as by design considerations.

### Applications

The present invention may be used for a number of different therapeutic applications, including but not limited to: (1) the post treatment closure and hemostasis of tissue tracks and vascular punctures following a percutaneous transcatheter procedure, tissue or organ biopsy procedure; (2) focal ablation of benign and malignant tumors, fibroids, and other tissue masses; (3) tissue tightening applications, including the treatment of female stress incontinence; (4) cosmetic applications such as venous valve tightening; and (5) as a technique for collagen or tissue enhancement or bulking.

As will be appreciated by those skilled in the art, the present invention may be modified to operate at various operational parameters and may be used to achieve the specific thermal objective, such as coagulation, cavitation, necrosis, etc. For example, for focal ablation applications, cavitation or necrosis parameters provided below may be used. TABLE A provides various examples of operating conditions that may be employed in conjunction with the methods, systems, and devices of the present invention to affect the specific, and desired, therapeutic thermal change. The information provided below is applicable for ultrasonically heating a 1 mm thick portion of tissue, for about 1 sec, but the specific type of tissue to be treated, frequency and exposure times will all influence the intensity best employed.

TABLE A

| | Operational Frequency | Operational Frequency |
|---|---|---|
| Tissue Effect | (4 MHz) | (8 MHz) |
| Tissue coagulation and | 300 W/cm2 | 150 W/cm2 |
| tightening (for | | |
| hemostasis applications) | | |
| Tissue necrosis | 900 W/cm2 | 450 W/cm2 |

It will be apparent and appreciated by those skilled in the art that various additions, modifications and improvements can be made without departing from the spirit and scope of the invention. Additionally, although individual features of the embodiments of the invention may be shown in some drawings and not in others, those skilled in the art will recognize that individual features of one embodiment of the invention can be combined with any or all the features of another embodiment. Accordingly, it is not intended that the invention be limited, except as by the appended claims.

What is claimed is:

1. A method for producing hemostasis, tissue closure, and/or vessel closure following a percutaneous medical procedure wherein an access device is introduced to a patient creating a passageway, said method comprising the step of:
a) inserting an introducer sheath transcutaneously into an artery, thereby creating the passageway and an arteriotomy;
b) inserting a catheter through the passageway and arteriotomy to perform a transcatheter procedure;
c) inserting a thermal delivery probe into the passageway;
d) ultrasonically interrogating a section of the passageway using pulsed Doppler;
e) advancing the thermal delivery probe down the passageway until the pulsed Doppler indicates that the probe is at a pre-determined distance from the arteriotomy;
f) emitting sufficient thermal energy to the arteriotomy site in order to raise native tissue temperatures; and
g) inducing tissue and/or blood coagulation at the site.

2. The method of claim 1, wherein the passageway was created in order to access a femoral, brachial or peripheral vessel.

3. The method of claim 2, wherein thermal delivery probe has an outer diameter of about 4-10 French.

4. The method of claim 3, wherein the emitted thermal energy is ultrasonically applied using a high frequency, high power output ultrasound transducer.

5. The method of claim 1, wherein the emitted thermal energy is ultrasonically applied using a high frequency, high power output ultrasound transducer.

6. The method of claim 4, wherein the high frequency, high power output ultrasound transducer is located at a distal end of the thermal delivery probe.

7. The method of claim 5, wherein the high frequency, high power output ultrasound transducer is located at a distal end of the thermal delivery probe.

8. The method of claim 4, wherein the high frequency, high power output ultrasound transducer is operated at about 6 MHz and output about 2 W/cm₂.

9. The method of claim 5, wherein the high frequency, high power output ultrasound transducer is operated at about 6 MHz and output about 2 W/cm₂.

10. A method for producing hemostasis and tissue closure following a percutaneous medical procedure wherein an access device is introduced to a patient creating a passageway, said method comprising the following step:
a) inserting an introducer sheath transcutaneously into an artery, thereby creating the passageway and an arteriotomy;
b) inserting a catheter through the passageway and arteriotomy to perform a transcatheter procedure;
c) inserting a ultrasound probe into the passageway;
d) ultrasonically interrogating a section of the passageway using pulsed Doppler;
e) advancing the thermal delivery probe down the passageway until the pulsed Doppler indicates that the probe is at a pre-determined distance from the arteriotomy;
f) emitting sufficient focused high intensity focused ultrasound energy to the arteriotomy site in order to raise native tissue temperatures; and
g) inducing tissue and/or blood coagulation at the site.

11. The method of claim 10, wherein thermal delivery probe has an outer diameter of about 2-7 French.

12. The method of claim 11, wherein the emitted thermal energy is ultrasonically applied using a high frequency, high power output ultrasound transducer.

13. The method of claim 10, wherein the emitted thermal energy is ultrasonically applied using a high frequency, high power output ultrasound transducer.

14. The method of claim 12, wherein the high frequency, high power output ultrasound transducer is located at a distal end of the thermal delivery probe.

15. The method of claim 13, wherein the high frequency, high power output ultrasound transducer is located at a distal end of the thermal delivery probe.

16. The method of claim 12, wherein the high frequency, high power output ultrasound transducer is operated at about 6 MHz and output about 2 W/cm₂.

17. The method of claim 13, wherein the high frequency, high power output ultrasound transducer is operated at about 6 MHz and output about 2 W/cm₂.

18. A therapeutic medical device adapted to be inserted into a tissue passageway following a percutaneous medical procedure, comprising:
a) an elongated shaft having a proximal section, a distal section, a distal tip, at least one lumen extending longitudinally from the distal tip to a proximal end located in the proximal section, a first opening in the proximal section in communication with the lumen, and a second opening on the distal tip in communication with the lumen, wherein the first and second openings and the lumen are configured such that the shaft may be threaded over a guidewire extending through the first opening and lumen and out the second opening;
b) a means for locating and determining a site at which thermal energy should be applied to promote hemostasis; and
c) a means for emitting sufficient thermal energy to the site thereby raising native tissue temperatures thereby inducing tissue and/or blood coagulation.

19. An insertable probe for delivering thermal energy comprising:
a) a elongated shaft having a proximal end, a distal end, at least one lumen extending longitudinally from said proximal end to said distal end, a first opening in the proximal section in communication with the lumen, and a second opening in the distal end in communication with the lumen, wherein the first and second openings and the lumen are configured such that the shaft may be threaded over a guidewire extending through the first opening and lumen and out the second opening; and
b) one or more ultrasound transducers positioned in the elongated shaft; said one or more ultrasound transducers comprising at least one therapeutic ultrasound transducer configured to emit high intensity ultrasound.

20. The insertable probe of claim 19, further comprising a diagnostic ultrasound transducer adapted to ultrasonically interrogate a position in front of the elongated shaft distal end.
